(19) United States
(12) Patent Application Publication
(10) Pub. No.: US 2026/0013905 A1
(43) Pub. Date: Jan. 15, 2026

(54) INTRA-OPERATIVE COMPRESSION DEVICE FOR INTRAMEDULLARY NAILING

(71) Applicant: DEPUY SYNTHES PRODUCTS, INC., Raynham, MA (US)

(72) Inventors: Stanley KMIEC, Morgantown, PA (US); Kevin CLANCY, West Chester, PA (US); Tom KEYER, West Chester, PA (US)

(21) Appl. No.: 19/247,670

(22) Filed: Jun. 24, 2025

Related U.S. Application Data
(60) Provisional application No. 63/668,971, filed on Jul. 9, 2024.

(51) Int. Cl. A61B 17/66; A61B 17/00; A61B 17/72

(52) U.S. Cl. CPC A61B 17/66; A61B 17/7216; A61B 2017/00367

(57) ABSTRACT

A system includes an intramedullary nail, a pin, a spindle, and a compression device including a pair of arms having distal and proximal ends. The nail is inserted through a medullary canal of a bone, extends longitudinally from a proximal end to a distal end and includes slotted opening extending transversely through a proximal portion of the nail and elongated along a length of the nail. The pin extends longitudinally from proximal to distal ends and is inserted through the opening. The spindle is releasably coupled to an insertion handle facilitating implantation of the nail. The distal ends connected to one another via a distal block releasably engaging the spindle. The proximal ends include engaging members mounted over the pin, so that moving the spindle distally with respect to the handle correspondingly moves the pin through the opening in a distal direction relative to the nail.

### PRIORITY CLAIM

This application claims priority to U.S. Provisional Patent Application Ser. No. 63/668,971 filed Jul. 9, 2024; the disclosure of which is incorporated herewith by reference.

### BACKGROUND

Tibiotalocalcaneal (TTC) arthrodesis is a procedure involving fusion of the ankle joint and the subtalar joint to alleviate pain resulting from, for example, arthritic hindfoot or ankle joint, and/or to correct joint deformities. During a TTC fusion procedure, a nail is inserted into a medullary canal of a tibia via a plantar aspect of the foot so that the nail extends through the calcaneus, talus, and tibial bones. While compression of the ankle joint (talocrural joint) and/or subtalar joint (talocalcaneal joint) is desired, it is often difficult to achieve. Currently, a surgeon may attempt to achieve compression via a manual manipulation of, for example, an insertion device via which the nail is inserted into the medullary canal and the gaps at the fracture or joints are reduced.

### SUMMARY

The present disclosure relates to a system for applying an intra-operative compression during treatment of bones. The system includes an intramedullary nail configured to be inserted through a medullary canal of a bone, the intramedullary nail extending longitudinally from a proximal end to a distal end and including slotted opening extending transversely through a proximal portion of the intramedullary nail and elongated along a length of the intramedullary nail; a pin extending longitudinally from a proximal end to a distal end, the pin sized and shaped to be inserted through the slotted opening of the intramedullary nail; a spindle configured to be releasably coupled to an insertion handle configured to facilitate implantation of the intramedullary nail; and a compression device including a pair of arms, distal ends of which are connected to one another via a distal block configured to releasably engage the spindle, and proximal ends of which include engaging members configured to be mountable over the pin, so that moving the spindle distally with respect to the insertion handle correspondingly moves the pin through the slotted opening in a distal direction relative to the intramedullary nail.

In an embodiment, the spindle includes a proximal portion configured to be releasably coupled to the insertion handle via a threading and a distal portion extending distally from the distal end of the insertion handle, when the proximal portion is coupled thereto, so that the distal block is engageable therewith.

In an embodiment, the distal portion of the spindle includes a shoulder configured to abut a proximal end of the distal block.

In an embodiment, the spindle is an assembly comprising an inner member configured to be releasably coupled to the insertion handle and an outer member mounted over the inner member so that the distal block of the compression device is engageable with the outer member, the outer member and the inner member configured to threadedly engage one another so that a rotation of the outer member relative to the inner member moves the inner and outer members longitudinally relative to one another.

In an embodiment, the proximal end of the distal block includes a counterbore that is sized, shaped, and configured to seat the shoulder therein.

In an embodiment, each of the distal ends of the pair of arms are pivotally connected to the distal block so that the pair of arms are movable relative to one another and relative to a longitudinal axis of the intramedullary nail, in an operative configuration.

In an embodiment, the distal block is defined via a first surface which, in an operative configuration, faces away from the insertion handle and, and a second surface which, in the operative configuration, faces toward the insertion handle, the second surface including a longitudinal groove extending therealong, the longitudinal groove sized, shaped and configured to receive a portion of the spindle therein.

In an embodiment, each of the engaging members is pivotally coupled to a corresponding one of the proximal ends of the pair of arms.

In an embodiment, each of the engaging members includes a groove extending transversely along a surface thereof, an opening of the groove sized, shaped, and configured to facilitate a mounting of the engaging member over the pin.

In an embodiment, a diameter of the groove is sized, shaped, and configured to receive a sleeve that is slidable over the pin.

In addition, the present disclosure relates to a system for applying an intra-operative compression. The system includes a spindle configured to be releasably coupled to a distal end of an insertion handle configured to insert an intramedullary nail such that the spindle is longitudinally movable relative to the insertion handle; and a compression device including a pair of arms, each of the pair of arms extending from a distal end toward a proximal end, the distal ends of the arms connected to one another via a distal block configured to engage a portion of the spindle, each of the proximal ends including an engaging member having a groove extending transversely therealong, the groove including an opening sized and shaped to facilitate mounting of the engaging members over a pin.

In an embodiment, the spindle includes a proximal portion configured to be releasably coupled to the insertion handle via a threading and a distal portion extending distally from the distal end of the insertion handle, when the proximal portion is coupled thereto, so that the distal block is engageable therewith.

In an embodiment, the spindle is an assembly including an inner member and an outer member, the inner member extending longitudinally from a proximal end configured to engage the insertion handle and the outer member mounted over the inner member such that the outer member is movable in a distal direction relative to the inner member.

In an embodiment, the outer member and the inner member are configured to threadedly engage one another so that a rotation of the outer member relative to the inner member moves the inner and outer members longitudinally relative to one another.

In an embodiment, the spindle includes a shoulder configured to abut the distal block so that a distal movement spindle relative to insertion handle correspondingly moves the compression device in a distal direction.

In an embodiment, each of the distal ends of the arms of the compression device are pivotally coupled to the distal block.

In an embodiment, each of the engaging members is pivotally coupled to a corresponding one of the proximal ends of the arms.

In an embodiment, the distal block includes a longitudinal groove extending therealong, the longitudinal groove being sized, shaped, and configured to receive the portion of the spindle therein.

In addition, the present disclosure relates to a method for treating bones. The method includes inserting an intramedullary nail through a medullary canal of a tibia, using an insertion handle, via a plantar aspect of a foot so that a distal portion of the intramedullary nail extends through a talus and calcaneus of the foot and a proximal portion extends through the tibia; inserting a fixation element through the calcaneus and a distal locking hole extending through the distal portion of the intramedullary nail; inserting a pin through a proximal end of a slotted hole extending transversely through the proximal portion and elongated along a length of the intramedullary nail; coupling a spindle to a distal end of the insertion handle; coupling a distal portion of a compression device to a portion the spindle and a proximal portion of the compression device to a portion of the pin extending from the tibia; and moving the spindle distally relative to the insertion handle to correspondingly move the pin distally through the slotted hole, thereby applying a compressive force to the tibia and ankle joints.

In an embodiment, the method further includes inserting the fixation element through a proximal locking hole extending through the proximal portion of the intramedullary nail to fix and maintain the compression of the ankle joints and fix the intramedullary nail relative to the tibia.

In an embodiment, coupling the spindle to the distal end of the insertion handle includes coupling an inner member to the insertion handle and mounting an outer member over the inner member so that the outer member is movable in a longitudinal direction relative thereto, and the distal portion of the compression device coupled to the outer member of the spindle.

In an embodiment, coupling the spindle to the distal end of the insertion handle includes threadedly engaging a proximal portion of the spindle with the distal end of the insertion handle so that a distal portion of the spindle extends distally therefrom to be coupled to distal portion of the compression device.

In an embodiment, the compression device includes a pair of arms, each of the pair of arms extending from a distal end toward a proximal end, the distal ends of the arms connected to one another via a distal block configured to engage the spindle, and each of the proximal ends including an engaging member having a groove extending transversely therealong, the groove including an opening sized and shaped to facilitate mounting of the engaging members over the pin.

In an embodiment, the pin is inserted through the tibia and the slotted hole so that a first portion of the pin extends from a first side of the tibia and a second portion of the pin extends from a second side of the tibia opposing the first side.

In an embodiment, the method further includes sliding a first sleeve over the first portion of the pin and through of a corresponding one of the engaging members; and sliding a second sleeve over the second portion of the pin and through the groove of a corresponding one of the engaging members.

### BRIEF DESCRIPTION

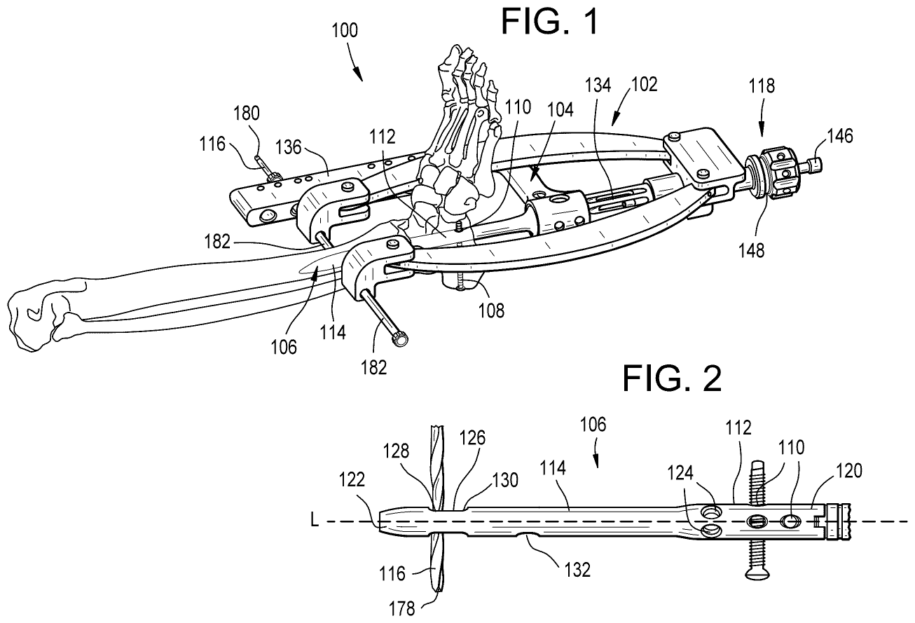

FIG. 1 shows a perspective view of a system according to an exemplary embodiment of the present disclosure;

FIG. 2 shows a longitudinal side view of an intramedullary nail according to the system of FIG. 1;

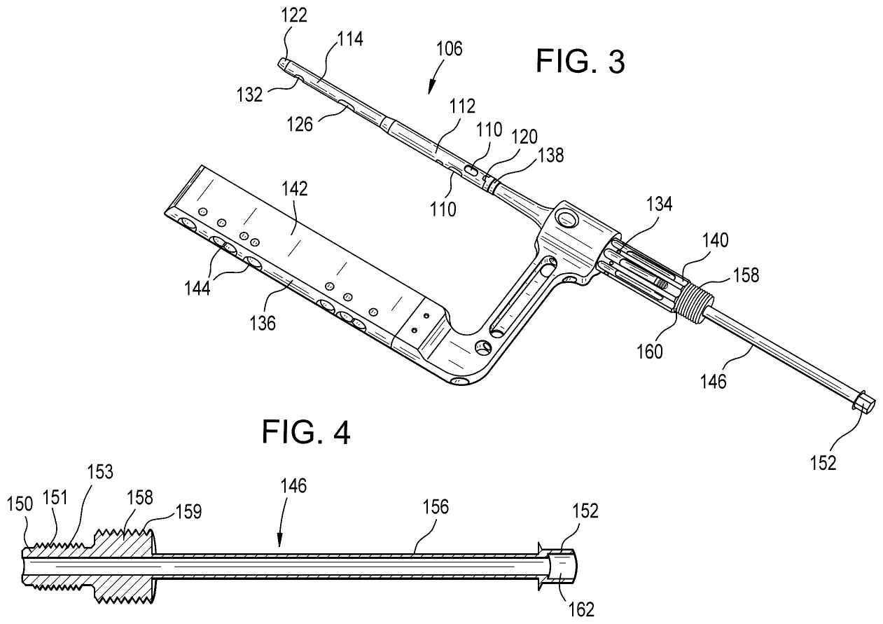

FIG. 3 shows a perspective view of an insertion device coupled with an inner spindle of a spindle assembly according to the system of FIG. 1;

FIG. 4 shows a longitudinal cross-sectional view of the inner spindle according to the system of FIG. 1;

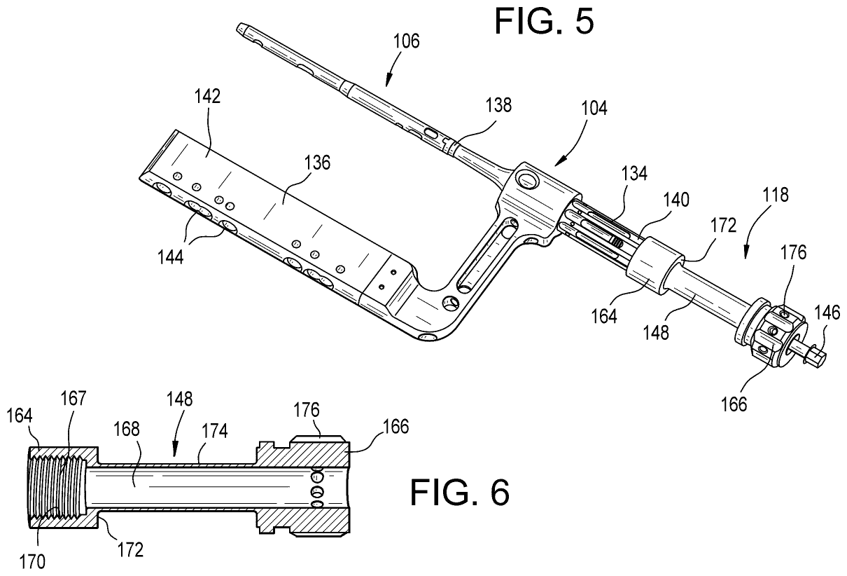

FIG. 5 shows a perspective view of the insertion device coupled with the spindle assembly according to the system of FIG. 1;

FIG. 6 shows a longitudinal cross-sectional view of an outer spindle of the spindle assembly according to the system of FIG. 1;

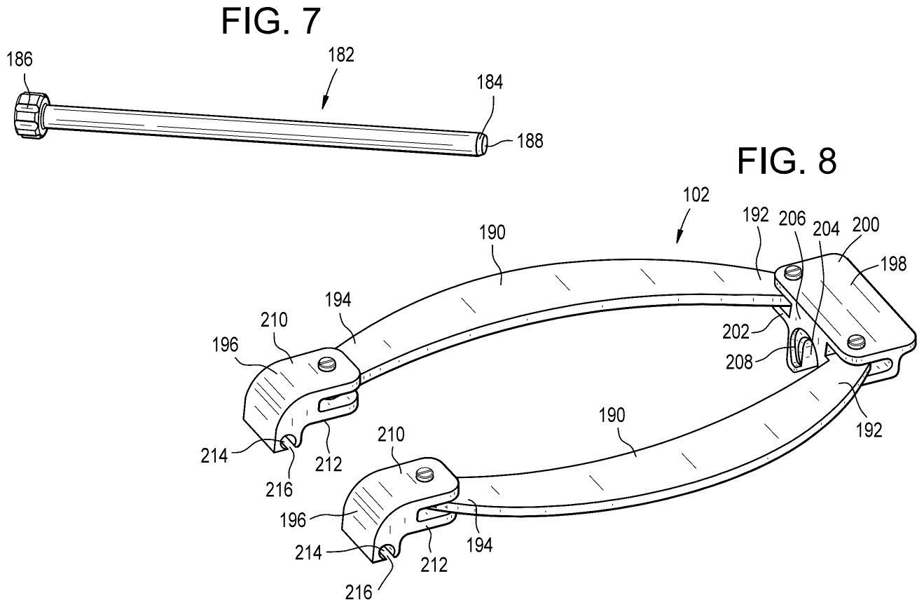

FIG. 7 shows a perspective view of a sleeve according to the system of FIG. 1;

FIG. 8 shows a perspective view of a compression device according to the system of FIG. 1;

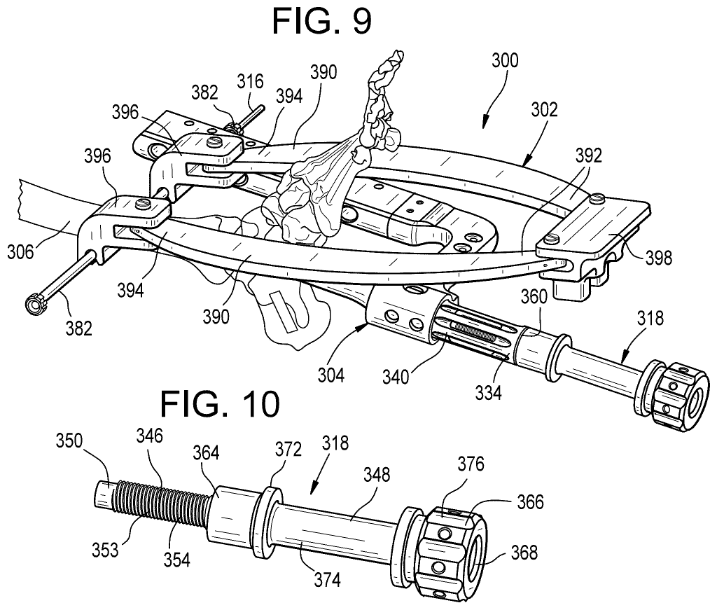

FIG. 9 shows a perspective view of a system according to another exemplary embodiment of the present disclosure; and

FIG. 10 shows a perspective view of a spindle according to the system of FIG. 9.

### DETAILED DESCRIPTION

The present disclosure may be further understood with reference to the following description and appended drawings, wherein like elements are referred to with the same reference numerals. The present disclosure relates to an intramedullary nail system and, in particular, a compression device configured to intra-operatively compress the talocrural and/or talocalcaneal joints. Exemplary embodiments of the present disclosure describe an intramedullary nail system including a compression device configured to be releasably coupled to an insertion device, via which an intramedullary nail (IM) nail is inserted into a medullary canal of a tibia during a TTC fusion procedure, to intra-operatively apply a compression to the talocrural and/or talocalcaneal joints.

It will be understood by those of skill in the art that the terms “proximal” and “distal,” as used herein, correspond to an anatomical orientation of the exemplary system, in an operative position. It will also be understood by those of skill in the art that although the exemplary embodiments are specifically shown and described with respect to a TTC fusion procedure, the system and method of the present disclosure may be utilized for the treatment of any of a variety of bones and/or joints in which a compression thereof would be desired.

As shown in FIGS. 1-8, an intramedullary nailing system 100 according to an exemplary embodiment of the present disclosure comprises a compression device 102 configured to be releasably coupled to an insertion device 104 via which an IM nail 106 is inserted through a medullary canal of a bone (e.g., tibia) to apply a compression along, for example, a talocrural and/or talocalcaneal joint during a TTC fusion procedure. According to an exemplary embodiment, the IM nail 106 may be inserted through a plantar aspect of a foot so that the IM nail 106 extends through a calcaneus, a talus, and the tibia. Upon insertion of the IM nail 106 into the bones, as desired, a fixation element 108 (e.g., screw) may be inserted through one or more distal locking holes 110, which extend transversely through a distal portion 112 of the IM nail 106 (e.g., portion of the IM nail 106 extending through a calcaneus) to fix the IM nail relative to the calcaneus.

As shown in FIG. 1, the compression device 102 may then be releasably coupled to the tibia and the insertion device 104. In an exemplary embodiment, the compression device 102 may be connected to the tibia via a pin 116 inserted through the tibia and a slotted hole 126 extending through a proximal portion 114 of the IM nail 106. The compression device 102 may be coupled to the insertion device 104, via a spindle assembly 118 connected to an end of the insertion device 104. As will be described in further detail below, the compression device 102 is releasably coupled to the insertion device 104 and the tibia so that compression device 102 may apply a compressive force to the tibia relative to the insertion device 104 and the IM nail 106, thereby compressing the talocrural and/or talocalcancal joints.

As shown in FIG. 2, the IM nail 106 extends along a longitudinal axis L from a distal end 120 to a proximal end 122. According to an exemplary embodiment, a proximal portion of the IM nail 106 is sized, shaped and configured to be inserted into a medullary canal of a tibia via a plantar aspect of the foot so that, in an operative position, the distal portion 112 is received within the calcaneus and talus while the proximal portion 114 is received within the tibia. In an exemplary embodiment, the distal portion 112 is configured to releasably engage a portion of the insertion device 104. In some embodiments, the distal portion 112 may have a diameter that is larger than a diameter of the proximal portion 114. The distal portion 112, however, is not required to have a larger diameter than the proximal portion 114. For example, the distal portion 112 may have a diameter of 13 mm while the diameter of the proximal portion 114 may have a diameter ranging from between 9 mm and 13 mm. It will be understood by those of skill in the art, however, that these dimensions are exemplary only and that the IM nail 106 may have any of a variety of sizes, shapes, and configurations.

The distal portion 112 of the IM nail 106 includes a plurality of distal locking holes 110, each of which extends along a central axis that extends transversely through the distal portion 112—angled with respect to the longitudinal axis L of the IM nail 106. In an exemplary embodiment, the central axis of each of the distal locking holes 110 extends substantially perpendicular relative to the longitudinal axis L of the IM nail 106. In an exemplary embodiment, two of the distal locking holes 110 extend through the distal portion 112, equally distanced from the distal end 120 but having central axes which extend substantially perpendicular to one another so that a user of the system 100 (e.g., surgeon) may insert a fixation element through a desired one of the distal locking holes 110, in a desired direction (e.g., lateral-medial or posterior-anterior). As described above, the distal locking holes 110 are positioned along the distal portion 112 of the IM nail 106 and configured so that, when the IM nail 106 is in an operative position, a fixation element (e.g., a locking screw) may be inserted through one or more of the distal locking holes 110 to fix the IM nail 106 relative to the calcaneus.

In an exemplary embodiment, the distal portion 112 also includes a talus locking hole 124 extending therethrough along a central axis extending transverse to the longitudinal axis L of the IM nail 106. The talus locking hole 124 may extend through the distal portion 112 proximally of the distal locking holes 110. In an exemplary embodiment, the central axis of the talus locking hole 124 may extend through the distal portion 112 at a non-perpendicular angle relative to the longitudinal axis of the IM nail 106. An angle of the central axis of the talus locking hole 124 may be selected so that, when the IM nail 106 is in the operative position, a fixation element inserted through the talus locking hole 124 extends through the talus bone in a desired configuration. Similarly to the distal locking holes 110, in another exemplary embodiment, the distal portion 112 may include two talus locking holes 124, each of which is equally distanced from the distal end 120 but where central axes of the two talus locking holes 124 are angled about the longitudinal axis L of the IM nail 106 so that they extend substantially perpendicular to one another. Thus, a user may select one of the talus locking holes 124 so that a fixation element (e.g., locking screw) inserted through the selected one of the talus locking holes 124 passes through the IM nail 106 in a desired direction.

The above description of the distal locking holes 110 and the talus locking holes 124 are illustrative and should not be viewed as limiting. It will be understood by those of skill in the art that the distal portion 112 of the IM nail 106 may have any number of locking holes extending therethrough in any of a variety of configurations so long as the IM nail 106 is configured to be fixed relative to the calcaneus and/or talus, as desired. In another exemplary embodiment, the distal portion 112 may include a locking hole extending transversely therethrough along a central axis configured such that, when a fixation element is inserted therealong, the fixation element extends across the subtalar joint—e.g., from the calcaneus, across the subtalar joint, and through the talus.

The proximal portion 114 includes the slotted hole 126 and at least one proximal locking hole. The slotted hole 126 extends through the proximal portion 114 along a central axis extending transverse to the longitudinal axis of the IM nail 106. The slotted hole 126 is elongated along a length of the proximal portion 114 and extends from a proximal end 128 to a distal end 130. Thus, as will be described in further detail below, the pin 116 may be inserted through the tibia and the proximal end 128 of the slotted hole 126 to apply a compressive force to the tibia. In particular, during the intra-operative compression, the tibia in a distal direction relative to the IM nail 106 so that the pin 116 slides from the proximal end 128 of the slotted hole 126 toward the distal end 130 of the slotted hole 126. In another exemplary embodiment, the proximal portion 114 may include a second slotted hole. It will be understood by those of skill in the art that the second slotted hole may be utilized for dynamization in situations where a bone area is compromised during compression via of the slotted hole 126.

The proximal portion 114 also includes a proximal locking hole 132 which extends along a central axis extending transversely through the proximal portion 114, at an angle relative to the longitudinal axis of the IM nail 106. The proximal locking hole 132 is configured to receive a fixation element therethrough so that, when the IM nail 106 is in the operative position, a fixation element inserted through the proximal locking hole 132 fixes the proximal portion 114 of the IM nail 106 relative to the tibia. A position of the proximal locking hole 132 along the proximal portion 114 may vary depending on, for example, a length of the IM nail 106. In some embodiments, the proximal locking hole 132 may extend through the proximal portion 114 proximally of the slotted hole 126. In another embodiment, the proximal locking hole 132 may extend through the proximal portion 114 distally of the slotted hole 126. Although the exemplary embodiment is shown and described as including a single proximal locking hole 132, it will be understood by those of skill in the art that the proximal portion 114 may include a plurality of proximal locking holes 132.

As will be understood by those of skill in the art, the IM nail 106 may have any of a variety of configurations so long as the IM nail 106 is configured to be inserted through a medullary canal of a bone to fix portions of the bone and or adjacent bones relative to one another. It will be understood by those of skill in the art that although the IM nail 106 is shown and described as having a specific configuration of holes (e.g., the proximal locking hole 132, the distal locking holes 110, and the talus locking hole 124), the IM nail 106 may include any of a variety of holes and/or openings in any of a variety of configurations where the holes and/or openings are configured to receive any of a variety of types of fixation devices.

According to an exemplary embodiment, the IM nail 106 is configured (sized, shaped, etc.) to be inserted into the medullary canal of the tibia via the insertion device 104 which includes an insertion handle 134 configured to facilitate an insertion of the IM nail 106 through the medullary canal of the bone and an aiming arm 136 configured to facilitate insertion of fixation elements through the IM nail 106.

The insertion handle 134 is configured to be releasably engaged with the distal end 120 of the IM nail 106, in longitudinal alignment therewith. According to an exemplary embodiment, the insertion handle 134 extends longitudinally from a proximal end 138 configured to engage the distal end 120 of the IM nail 106 to a distal end 140 that, as will be described in further detail below, is configured to engage the spindle assembly 118. The distal end 140 may include, for example, a threading along an interior thereof. In an exemplary embodiment, the insertion handle 134 is configured to facilitate driving of the IM nail 106 into the medullary canal of the tibia via, for example, manual manipulation and/or hammering as would be understood by those skilled in the art.

In an exemplary embodiment, the aiming arm 136 is configured to be coupled to the insertion handle 134 so that an aiming block 142 of the aiming arm 136 extends substantially parallel to the longitudinal axis of the IM nail 106 in an operative configuration. The aiming block 142 includes a plurality of openings 144 extending therethrough with each of the openings 144 extending along a central axis. Each of the openings 144 is positioned and oriented so that, when the aiming arm 136 is in an operative configuration, each opening 144 is aligned with a corresponding one of the distal locking holes 110, the talus locking hole 124, the slotted hole 126, and the proximal locking hole 132. As will be understood by those of skill in the art, the aiming arm 136 is movable relative to the insertion handle 134 to align one or more of the openings 144 with a desired one of the holes of the IM nail 106. Thus, instruments such as, for example, a protection sleeve, a drill sleeve, pins, fixation elements, drills, etc., may be passed through an opening 144 of the aiming block 142 to a desired one of the holes (e.g., the distal locking holes 110, the talus locking hole 124, the slotted hole 126, and the proximal locking hole 132) of the IM nail 106.

In particular, during a procedure requiring intra-operative compression of the talocrural and/or talocalcaneal joints according to an exemplary embodiment, the pin 116 is guided through a first one of the openings 144 that is in alignment with the proximal end 128 of the slotted hole 126 so that the pin 116 passes through the first one of the openings 144 through an intervening portion of bone and into the slotted hole 126. Although not shown or described, it will be understood by those of skill in the art that the pin 116 may be guided to the proximal end 128 of the slotted hole 126 via, for example, a protective sleeve previously inserted through the corresponding opening 144.

It will be understood by those of skill in the art that although the system 100 is described with respect to the insertion device 104 shown in the figures, the system 100 may be utilized with any of a variety of insertion devices 104 so long as the insertion device 104 includes an insertion handle 134 configured to be coupled to the compression device 102, as described herein. For example, the aiming arm 136 may have any of a variety of configurations and although described as being movable and releasably coupled to the insertion handle 134, may be fixed to the insertion handle 134. In addition, the aiming arm 136 may include any of a variety of differently sized, shaped, and configured openings 144 extending therethrough so long as they are, in an operative configuration, in alignment with one or more of the holes of the IM nail 106.

In an exemplary embodiment, the spindle assembly 118 includes an inner spindle 146 configured to releasably engage the distal end 140 of the insertion handle 134 and an outer spindle 148 configured to be mounted thereover. The inner spindle 146 extends longitudinally from a proximal end 150 to a distal end 152. The proximal end 150 includes a connecting portion 151 including, for example, a threading 153 along an exterior surface 154 thereof. In an exemplary embodiment, the connecting portion 151 is configured to be threadedly received within the distal end 140 of the insertion handle 134. It will be understood by those of skill in the art that although the connecting portion 151 is shown and described as including a threading 153, the connecting portion 151 may include any of a variety of engaging structures configured to releasably couple the proximal end 150 of the inner spindle 146 with the distal end 140 of the insertion handle 134.

According to an exemplary embodiment, an abutment 158 extends along a portion of the inner spindle 146 distal of a connecting feature (e.g., threading 153) so that upon engagement of the connecting feature with the distal end 140 of the insertion handle 134, the abutment 158 extends distally of the distal end 140. In an exemplary embodiment, the abutment 158 has a larger diameter than the connecting portion 151 and a remaining length 156 of the inner spindle 146 so that, when the inner spindle 146 is connected to the insertion handle 134 in the operative configuration, the abutment 158 abuts or contacts a distal face 160 thereof. Although the abutment 158 of this embodiment includes a threading 159 as the connecting feature, as would be understood by those skilled in the art, the connecting feature may be formed on a portion of the exterior surface 154 as any known structure suitable for engaging/coupling to the outer spindle 148, as will be described in further detail below.

The distal end 152 of the inner spindle 146 includes a coupling element 162 which is configured to engage a driver (not shown) for driving the connecting portion 151 of the proximal end 150 of the inner spindle 146 into engagement with the distal end 140 of the insertion handle 134. In an exemplary embodiment, the coupling element 162 includes a recess sized, shaped and configured (e.g., hex-shaped recess) to engage a correspondingly shaped tip of the driver so that rotation of the driver correspondingly rotates the inner spindle 146 facilitating threaded engagement between the connecting portion 151 and the distal end 140 of the insertion handle 134. Although the coupling element 162 is shown and described as a recess, it will be understood by those of skill in the art that the engaging element 162 may have any of a variety of configurations so long as the coupling element 162 is engageable with a correspondingly sized and shaped portion of the driver. In another embodiment, for example, the coupling element 162 may be defined via an exterior that is sized and shaped to be received within a corresponding portion of the driver.

The outer spindle 148 extends longitudinally from a proximal end 164 to a distal end 166 and includes a channel 168 extending therethrough. The channel 168 is sized and shaped to slidably receive a portion of the inner spindle 146 therein so that the outer spindle 148 may be mounted thereover. In an exemplary embodiment, the proximal end 164 is configured to extend over the abutment 158 and includes threading 167 extending along a portion of an interior surface 170 where the threading 167 corresponds to the threading 159 of the abutment 158 so that the inner and outer spindles 146, 148 may be threadedly engaged with one another. Upon assembly of the outer spindle 148 over the inner spindle 146, the proximal end 164 of the outer spindle 148 abuts the distal face 160 of the insertion handle 134. It will be understood by those of skill in the art that although the distal end 166 of the outer spindle 148 is shown and described as being connectable with the abutment 158 of the inner spindle 146 via a threaded connection, the inner and outer spindles 146, 148 may be configured to be coupled to one another via any of a variety of engagement mechanisms.

In an exemplary embodiment, the outer spindle 148 also includes a shoulder 172 along an exterior 174 thereof and facing distally. As will be described in further detail below, the shoulder 172 is configured to abut/engage a portion of the compression device 102 so that when the outer spindle 148 is moved longitudinally relative to the inner spindle 146 via, for example, rotation relative thereto, the compression device 102 is correspondingly moved longitudinally (e.g., in a distal direction) relative to the inner spindle 146 and the IM nail 106 to apply a compressive force to the bone via the pin 116 inserted through the bone and the IM nail 106.

In an exemplary embodiment, the distal end 166 of the outer spindle 148 includes a thumb wheel 176 configured so that, when rotated by the user, the thumb wheel correspondingly rotates the outer spindle 148 relative to the inner spindle 146. The thumb wheel 176 according to an exemplary embodiment includes an optional feature facilitating gripping of the thumb wheel 176 by the user to facilitate rotation of the outer spindle 148. In an exemplary embodiment, a distance between the thumb wheel 176 and the shoulder 172 is configured to accommodate a portion of the compression device 10 therebetween.

Although the exemplary embodiments show and describe the outer spindle 148 that is rotatable relative to the inner spindle 146 to facilitate compression via the compression device 102, it will be understood by those of skill in the art that the spindle assembly 118 may have any of a variety of configurations so long as the spindle assembly 118 is configured to facilitate distal movement of the compression device 102 when it is coupled thereto. In another embodiment, for example, rather than the outer spindle 148, the spindle assembly 118 may include a cam mechanism configured to move distally relative to the inner spindle 146 to correspondingly move the compression device 102. In yet another embodiment, the spindle assembly 118 may include a lever arm for moving the compression device 102.

As described above, the pin 116 is sized and configured to be inserted through an intervening portion of the bone (i.e., a portion of the bone between the aiming arm 136 and the IM nail 106) and the slotted hole 126. In particular, the pin 116 extends longitudinally from a first end 178 to a second end 180 so that when the pin 116 is inserted transversely through the bone, in an operative position, the first end 178 projects out of a first side of the bone while the second end 180 projects toward the aiming arm 136 from a second side of the bone opposing the first side. In an exemplary embodiment, the system 100 further comprises a pair of sleeves 182, each of which is sized, shaped, and configured to be slid over opposing portions of the pin 116 projecting out of the bone after they have been inserted in the operative position. Each of the sleeves 182 extends longitudinally from a first end 184 to a second end 186 and includes a channel 188 extending therethrough that is sized, shaped, and configured to slidably receive the corresponding portion of the pin 116 therein.

According to an exemplary embodiment, the compression device 102 includes a pair of arms 190 extending from distal ends 192 to proximal ends 194. The distal ends 192 of which are connected to one another via a distal block 198 that is sized, shaped, and configured to engage the shoulder 172 of the outer spindle 148. The proximal ends 194 of the arms 190 include engagement members 196, each of which is configured to releasably engage the pin 116. As will be described in further detail below, the compression device 102 engages the outer spindle 148 and the pin 116 such that the distal block 198 abuts the shoulder 172 so that longitudinal movement of the outer spindle 148 relative to the inner spindle 146 correspondingly moves the compression device 102.

In an exemplary embodiment, each of the arms 190 extends from its distal end 192 to a proximal end 194 along a curve configured so that, when the compression device 102 is coupled to the insertion device 104 in the operative configuration, the arms 190 curve away from a longitudinal axis of the compression device 102 to extend along either side of the foot without interfering therewith. Each of the distal ends 192 is pivotally connected to the distal block 198 which, according to an exemplary embodiment, is sized shaped and configured to be received between the thumb wheel 176 and the shoulder 172 of the outer spindle 148. Although the arms 190 are shown and described as extending along a curve, it will be understood by those of skill in the art that the arms 190 may have any of a variety of configurations so long as the arms 190 are shaped to extend about the foot—i.e., extend along either side of the foot—without contacting the foot and/or interfering with other anatomical areas.

In an exemplary embodiment, the distal block 198 includes a first surface 200 which, when the compression device 102 is in the operative configuration, faces away from the insertion device 104, and a second surface 202 which, when the compression device 102 is in the operative configuration, faces toward the insertion device 104. Each of the distal ends 192 is pivotally coupled to the distal block 198 and received between the first and second surfaces 200, 202 such that the arms 190 are movable relative to one another, toward and away from the longitudinal axis of the compression device 102. The second surface 202 includes a longitudinal groove 204 extending therealong (i.e., along the longitudinal axis of the compression device 102), wherein the longitudinal groove 204 is sized, shaped, and configured to receive a portion of the outer spindle 148 therein.

In particular, in an exemplary embodiment, the longitudinal groove 204 is configured to receive a length of the outer spindle 148 extending between the shoulder 172 and the thumb wheel 176. A proximal face 199 of the distal block 198 may include a counterbore 208 sized, shaped, and configured to seat the shoulder 172 of the outer spindle 148 therein to prevent inadvertent movement of the compression device 102 relative to the outer spindle 148 during a compression procedure. As the distal block 198 abuts and/or engages the shoulder 172 of the compression device 102, longitudinal movement of the outer spindle 148 relative to the inner spindle 146 correspondingly moves the compression device 102 relative to the IM nail 106.

According to an exemplary embodiment, each of the engagement members 196 is pivotally coupled to a corresponding one of the proximal ends 194 of the arms 190. Similarly to the distal block 198, each of the engagement members 196 is defined via a first surface 210 which, when the compression device 102 is in the operative configuration, faces away from the pin 116, and a second surface 212 which, in the operative configuration, faces toward the pin 116. The proximal ends 194 are pivotally received between the first and second surfaces 210, 212.

In an exemplary embodiment, the second surface 212 of each of the engagement members 196 includes a groove 214 extending therealong where the groove 214 sized, shaped, and configured to receive a portion of the pin 116 therein. Thus, when the compression device 102 is in the operative configuration, the groove 214 extends transverse to the longitudinal axis of the compression device 102 (i.e., a proximal-distal axis). In an exemplary embodiment, an opening 216 extending along a length of the groove 214 is sized to permit mounting of the engagement members 196 over the pin 116 while a diameter of the groove 214 is sized and shaped to slidably accommodate a corresponding one of the sleeves 182 therein. Thus, as will be described in further detail below, upon mounting of the engagement members 196 over the pin 116, each of the sleeves 182 may be slid along a corresponding portion of the pin 116, through the groove 214. It will be understood by those of skill in the art that receipt of the sleeves 182 over the pin 116 and through the grooves 214 of the engagement members 196 will aid in stabilizing the compression device 102 relative to the insertion device 104 and the bone.

According to another exemplary embodiment, rather than having sleeves 182 slidably received over the pin 116 and within the grooves 214 of the engaging members 196, the engaging members 196 may include protrusions extending therefrom, in alignment with the longitudinal grooves 204. In this embodiment, the grooves are sized, shaped, and configured to mountably engage the pin 116 via the opening 206 thereof. Upon mounting the engaging members 196 over the pin 116, the engaging members 196 may be moved toward one another until the protrusions which extend therefrom come into contact with the bone (e.g., tibia), stabilizing the compression device 102 relative thereto.

According to an exemplary method utilizing the system 100, the IM nail 106 is inserted into a medullary canal of a tibia a plantar aspect of a foot via an insertion handle 134 so that the distal portion 112 of the IM nail 106 extends through the talus and calcaneus while the proximal portion 114 of the IM nail 106 extends through the tibia. Upon insertion of the IM nail 106 into the medullary canal of the tibia, the IM nail 106 may be fixed relative to the calcaneus via one or more fixation elements 108 (e.g., locking screws) inserted through one or more of the distal locking holes 110. As will be understood by those of skill in the art, insertion of the one or more fixation element 108 through the distal locking holes 110 may be facilitated via the aiming arm 136 of the insertion device 104 as indicated above.

To assemble the compression device 102 with the tibia and the distal block 198, the pin 116 is inserted through the proximal end 128 of the slotted hole 126 and the spindle assembly 118 and connected to the distal end 140 of the insertion handle 134. According to an exemplary embodiment, the pin 116 is inserted through the tibia and the slotted hole 126 so that the first end 178 of the pin 116 projects from a first side of the tibia while a second end 180 of the pin 116 projects from a second side of the tibia opposing the first side. As described above with respect to the spindle assembly 118, the connecting portion 151 of the inner spindle 146 is then threadedly engaged with the distal end 140 of the insertion handle 134 and the outer spindle 148 is threadedly mounted over the inner spindle 146 so that the outer spindle 148 is rotatable relative to the inner spindle 146.

The compression device 102 is releasably coupled to the pin 116 and the spindle assembly 118 by first mounting the engaging members 196 over the pin 116 through the opening 216 of the grooves 214 therealong. In particular, a first one of the engaging members 196 is mounted over a portion of the pin 116 projecting from the first side of the tibia while a second one of the engaging members 196 is mounted over a portion of the pin 116 projecting from the second side of the tibia. Once the engaging members 196 have been releasably coupled to the pin 116, each of the sleeves 182 may be slid along a corresponding portion of the pin 116, through a corresponding one of the grooves 214 of the engaging members 196 until first ends 184 of the sleeves 182 contact the first and second sides of the tibia.

Once the engaging members 196 have been coupled to the pin 116, the compression device 102 is pivoted about the pin 116 to move the distal block 198 toward the spindle assembly 118. The distal block 198 is positioned over the outer spindle 148, distally of the shoulder 172 until the outer spindle 148 is received within the longitudinal groove 204 along the second surface 202 thereof. It will be understood by those of skill in the art that since each of the engaging members 196 (and the distal block 198) is pivotally coupled to the proximal ends 194 and the distal ends 192 of the arms 190, respectively, the engaging members 196 are movable along the pin 116—while maintaining an engagement and alignment therewith—as the distal block 198 is positioned over the outer spindle 148. Upon positioning the distal block 198 over the outer spindle 148, the shoulder 172 of the outer spindle 148 is seated within the counterbore 308 of the distal block 198 to prevent movement between the compression device 102 and the outer spindle 148.

After assembly of the compression device 102 with the insertion device 104, compression of the talocrural and/or talocalcaneal joints may be initiated. In an exemplary embodiment, the outer spindle 148 is moved distally relative to the inner spindle 146, via a rotation of the outer spindle 148 relative to the inner spindle 146, to correspondingly move the compression device 102 longitudinally relative to the IM nail 106. In particular, since the compression device 102 is engaged with the pin 116 that has been inserted through the tibia, distal movement of the compression device 102 correspondingly moves the tibia distally with respect to the proximal portion 114 of the IM nail 106 to compress the talocrural and/or talocalcaneal joints. As the tibia is moved distally relative to the IM nail 106, the pin 116 is slid from the proximal end 128 of the slotted hole 126 toward the distal end 130 thereof.

As will be understood by those of skill in the art, once the talocrural and/or talocalcaneal joints have been compressed to a desired degree, a first fixation element may be inserted through the talus locking hole 124 to fix the IM nail 106 relative to the talus and a second fixation element may be inserted through the proximal locking hole 132 to fix the IM nail 106 relative to the tibia.

Although the exemplary embodiments show and describe a compression device 102 including a pair of compression arms 190, it will be understood by those of skill in the art that the compression of the ankle joint and the subtalar joint may be similarly achieved via a compression device having a single arm. Rather than having the arms 190 extending along opposing sides of the bone, such a compression device may be releasably coupled to a portion of the insertion device 104 so that the arm extends between, for example, the aiming arm 136 and the IM nail 106.

It will be understood by those of skill in the art that a single arm 190 would be capable of connection to the insertion device 104 and the pin 116 in a manner substantially similar to that described above to apply compressive force to a bone to which it is releasably coupled. It will be understood by those of skill in the art that where there is only the single arm 190, the single arm 190 may not be required to be pivotally connected to the distal block 198 and/or the engaging member 196 so long as a length of the arm 190 may be adjusted to engage both the pin 116 and the spindle assembly 118 or other mechanism via which a movement of the compression device in a distal direction relative to the IM nail 106 may be facilitated.

As shown in FIGS. 9-10, an intramedullary nail system 300 may be substantially similar to the system 100 described above, comprising a compression device 302 configured to be releasably coupled to an insertion device 304 via which an IM nail 306 is inserted through a medullary canal of a bone to apply a compression during, for example, a TTC fusion procedure. As shown in FIG. 9, the IM nail 306 and the insertion device 304 may be substantially similar to the IM nail 106 and the insertion device 104 described above with respect to the system 100, the IM nail 306 configured to be similarly inserted through the medullary canal of the bone so that a pin 316 may be inserted transversely through the bone and a slotted hole (not shown) extending through a proximal portion of the IM nail 306. Similarly to the system 100, the compression device 302 may be coupled to the bone (via the pin 316) and the insertion device 304 to apply a compressive force across the talocrural and/or talocalcaneal joints. The compression device 302 may also be substantially similar to the compression device 102. Rather than being coupled to the insertion device 304 via a spindle assembly including inner and outer spindles, however, the compression device 302 is coupled to the insertion device 304 via a single spindle 318.

As shown in FIG. 10, the spindle 318 extends longitudinally from a proximal end 350 to a distal end 366 and includes a channel 368 extending therethrough. The spindle 318 includes a proximal portion 346 configured to engage a distal end 340 of an insertion handle 334 of the insertion device 304 and a distal portion 348 configured to extend distally of the distal end 340 of the insertion device 304, when the proximal portion 346 is couped to the insertion handle 134. The distal portion 348 is configured to be coupled to the compression device 302.

According to an exemplary embodiment, the proximal portion 346 may be configured to be received within the distal end 340 of the insertion handle 334 (see FIG. 9). In an exemplary embodiment, the proximal portion 346 may include a threading 353 along an exterior surface 354 thereof, the threading 353 configured to threadedly engage a corresponding threading along an interior of the distal end 340. The proximal portion 346 may have a smaller diameter than the distal portion 348 so that the proximal portion 346 may be threaded into the distal end 340 of the insertion handle 334 until a proximal end 364 of the distal portion 348 abuts a distal face 360 of the distal end 340. Although the proximal portion 346 is shown and described as including the threading 353, it will be understood by those of skill in the art that the proximal portion 346 may include any of a variety of engaging structures configured to releasably couple the proximal portion 346 with the distal end 340 of the insertion handle.

In an exemplary embodiment, the distal portion 348 may be substantially similar to the outer spindle 148 of the spindle assembly 118 described above with respect to the system 100. In particular, the distal portion 348 extends from the proximal end 364 thereof to the distal end 366. The distal portion 348 includes a shoulder 372 along an exterior 374 thereof and facing distally. As described above with respect to the system 100, the shoulder 372 is configured to abut/engage a portion of the compression device 302 (e.g., a distal block 398) so that, when the spindle 318 is moved longitudinally relative to the insertion handle 334 via, for example, rotation relative thereto, the compression device 302 is correspondingly moved longitudinally (e.g., in a distal direction) relative to the insertion handle 334 and the IM nail 306 to apply a compressive force to the bone via the pin 316 inserted through the bone and the IM nail 306.

In particular, according to an exemplary embodiment, the spindle 318 may be rotated to move the spindle 318 distally relative to the insertion handle 334 by unthreading the proximal portion 346 of the spindle 318 therefrom. Rotation of the spindle 318 may be facilitated via, for example, a thumb wheel 376 at the distal end 366 thereof, which is configured so that, when rotated by the user, the thumb wheel 376 correspondingly rotates the spindle 318 relative to the insertion handle 334. It will be understood by those of skill in the art that a distance between the thumb wheel 376 and the shoulder 372 is configured to accommodate the distal block 398 of the compression device 302 therebetween.

It will be understood by those of skill in the art that the system 300 may be utilized in a manner substantially similarly to the system 100 described above. To apply compression upon insertion of the IM nail 306 via the insertion handle 334 of the insertion device 304, the pin 316 is inserted through a proximal end of the slotted hole 326 and the spindle 318 is coupled to the distal end 340 of the insertion handle 334, as described above. The compression device 302 may be substantially similar to the compression device 102, comprising arms 390, distal ends 392 of which are connected to one another via the distal block 398 and proximal ends 394 of each of which are pivotally connected to an engagement member 396. Thus, similarly to the compression device 102, the compression device 302 is coupled to the pin 316 by mounting the engagement members 396 over the pin 316 on opposing sides of the bone so that the compression device 302 may be pivoted about the pin 316 until the distal block 398 is engaged with the distal portion 348 of the spindle 318. Although not explicitly described with respect to the system 300, it will be understood by those of skill in the art that the system 300 may also comprise sleeves 382, which substantially similarly to the sleeves 182 of the system 100, may be slid over opposing portions of the pin 316.

As described above, to apply a compressive force to the bone, the spindle 318 is rotated relative to the insertion handle 334 so that the spindle 318 is moved distally relative to the insertion handle 334. Since the distal block 398 abuts/engages the shoulder 372 of the spindle 318, rotation of the spindle 318 correspondingly moves the compression device 302 distally relative to the IM nail 306 to compress the talocrural and/or talocalcaneal joints.

It will be appreciated by those skilled in the art that changes may be made to the embodiments described above without departing from the inventive concept thereof. It should further be appreciated that structural features and methods associated with one of the embodiments can be incorporated into other embodiments. It is understood, therefore, that this invention is not limited to the particular embodiment disclosed, but rather, modifications are also covered within the scope of the present invention as defined by the appended claims.

What is claimed is:

1. A system for applying an intra-operative compression during treatment of bones, comprising:
an intramedullary nail configured to be inserted through a medullary canal of a bone, the intramedullary nail extending longitudinally from a proximal end to a distal end and including slotted opening extending transversely through a proximal portion of the intramedullary nail and elongated along a length of the intramedullary nail;
a pin extending longitudinally from a proximal end to a distal end, the pin sized and shaped to be inserted through the slotted opening of the intramedullary nail;
a spindle configured to be releasably coupled to an insertion handle configured to facilitate implantation of the intramedullary nail; and
a compression device including a pair of arms, distal ends of which are connected to one another via a distal block configured to releasably engage the spindle, and proximal ends of which include engaging members configured to be mountable over the pin, so that moving the spindle distally with respect to the insertion handle correspondingly moves the pin through the slotted opening in a distal direction relative to the intramedullary nail.

2. The system of claim 1, wherein the spindle includes a proximal portion configured to be releasably coupled to the insertion handle via a threading and a distal portion extending distally from the distal end of the insertion handle, when the proximal portion is coupled thereto, so that the distal block is engageable therewith.

3. The system of claim 2, wherein the distal portion of the spindle includes a shoulder configured to abut a proximal end of the distal block.

4. The system of claim 1, wherein the spindle is an assembly comprising an inner member configured to be releasably coupled to the insertion handle and an outer member mounted over the inner member so that the distal block of the compression device is engageable with the outer member, the outer member and the inner member configured to threadedly engage one another so that a rotation of the outer member relative to the inner member moves the inner and outer members longitudinally relative to one another.

5. The system of claim 3, wherein the proximal end of the distal block includes a counterbore that is sized, shaped, and configured to seat the shoulder therein.

6. The system of claim 1, wherein each of the distal ends of the pair of arms are pivotally connected to the distal block so that the pair of arms are movable relative to one another and relative to a longitudinal axis of the intramedullary nail, in an operative configuration.

7. The system of claim 1, wherein the distal block is defined via a first surface which, in an operative configuration, faces away from the insertion handle and, and a second surface which, in the operative configuration, faces toward the insertion handle, the second surface including a longitudinal groove extending therealong, the longitudinal groove sized, shaped and configured to receive a portion of the spindle therein.

8. The system of claim 1, wherein each of the engaging members is pivotally coupled to a corresponding one of the proximal ends of the pair of arms.

9. The system of claim 1, wherein each of the engaging members includes a groove extending transversely along a surface thereof, an opening of the groove sized, shaped, and configured to facilitate a mounting of the engaging member over the pin.

10. The system of claim 9, wherein a diameter of the groove is sized, shaped, and configured to receive a sleeve that is slidable over the pin.

11. A system for applying an intra-operative compression, comprising:
a spindle configured to be releasably coupled to a distal end of an insertion handle configured to insert an intramedullary nail such that the spindle is longitudinally movable relative to the insertion handle; and
a compression device including a pair of arms, each of the pair of arms extending from a distal end toward a proximal end, the distal ends of the arms connected to one another via a distal block configured to engage a portion of the spindle, each of the proximal ends including an engaging member having a groove extending transversely therealong, the groove including an opening sized and shaped to facilitate mounting of the engaging members over a pin.

12. The system of claim 11, wherein the spindle includes a proximal portion configured to be releasably coupled to the insertion handle via a threading and a distal portion extending distally from the distal end of the insertion handle, when the proximal portion is coupled thereto, so that the distal block is engageable therewith.

13. The system of claim 11, wherein the spindle is an assembly including an inner member and an outer member, the inner member extending longitudinally from a proximal end configured to engage the insertion handle and the outer member mounted over the inner member such that the outer member is movable in a distal direction relative to the inner member.

14. The system of claim 13, wherein the outer member and the inner member are configured to threadedly engage one another so that a rotation of the outer member relative to the inner member moves the inner and outer members longitudinally relative to one another.

15. The system of claim 11, wherein the spindle includes a shoulder configured to abut the distal block so that a distal movement spindle relative to insertion handle correspondingly moves the compression device in a distal direction.

16. The system of claim 11, wherein each of the distal ends of the arms of the compression device are pivotally coupled to the distal block.

17. The system of claim 11, wherein each of the engaging members is pivotally coupled to a corresponding one of the proximal ends of the arms.

18. The system of claim 11, wherein the distal block includes a longitudinal groove extending therealong, the longitudinal groove being sized, shaped, and configured to receive the portion of the spindle therein.

19. A method for treating bones, comprising:
inserting an intramedullary nail through a medullary canal of a tibia, using an insertion handle, via a plantar aspect of a foot so that a distal portion of the intramedullary nail extends through a talus and calcaneus of the foot and a proximal portion extends through the tibia;
inserting a fixation element through the calcaneus and a distal locking hole extending through the distal portion of the intramedullary nail;
inserting a pin through a proximal end of a slotted hole extending transversely through the proximal portion and elongated along a length of the intramedullary nail;
coupling a spindle to a distal end of the insertion handle;
coupling a distal portion of a compression device to a portion the spindle and a proximal portion of the compression device to a portion of the pin extending from the tibia; and
moving the spindle distally relative to the insertion handle to correspondingly move the pin distally through the slotted hole, thereby applying a compressive force to the tibia and ankle joints.

20. The method of claim 19, further comprising:
inserting the fixation element through a proximal locking hole extending through the proximal portion of the intramedullary nail to fix and maintain the compression of the ankle joints and fix the intramedullary nail relative to the tibia.

21. The method of claim 19, wherein coupling the spindle to the distal end of the insertion handle includes coupling an inner member to the insertion handle and mounting an outer member over the inner member so that the outer member is movable in a longitudinal direction relative thereto, and the distal portion of the compression device coupled to the outer member of the spindle.

22. The method of claim 19, wherein coupling the spindle to the distal end of the insertion handle includes threadedly engaging a proximal portion of the spindle with the distal end of the insertion handle so that a distal portion of the spindle extends distally therefrom to be coupled to distal portion of the compression device.

23. The method of claim 19, wherein the compression device includes a pair of arms, each of the pair of arms extending from a distal end toward a proximal end, the distal ends of the arms connected to one another via a distal block configured to engage the spindle, and each of the proximal ends including an engaging member having a groove extending transversely therealong, the groove including an opening sized and shaped to facilitate mounting of the engaging members over the pin.

24. The method of claim 23, wherein the pin is inserted through the tibia and the slotted hole so that a first portion of the pin extends from a first side of the tibia and a second portion of the pin extends from a second side of the tibia opposing the first side.

25. The method of claim 24, further comprising:
sliding a first sleeve over the first portion of the pin and through of a corresponding one of the engaging members; and
sliding a second sleeve over the second portion of the pin and through the groove of a corresponding one of the engaging members.
